(19) United States
(12) Patent Application Publication
(10) Pub. No.: US 2004/0157954 A1
(43) Pub. Date: Aug. 12, 2004

(54) Bone cement composition

(76) Inventors: Yohji Imai, Chiba (JP); Yoshinori Kadoma, Tokyo (JP); Kazuo Takakuda, Tokyo (JP); Sadao Morita, Saitama (JP)

Correspondence Address: AMIN & TUROCY, LLP, 1900 EAST 9TH STREET, NATIONAL CITY CENTER, 24TH FLOOR,, CLEVELAND, OH, 44114, US

(21) Appl. No.: 10/770,789

(22) Filed: Feb. 3, 2004

(30) Foreign Application Priority Data
Feb. 4, 2003 (JP) 2003-26948

(51) Int. Cl. A61F 2/00

(52) U.S. Cl. 523/115

(57) ABSTRACT

The bone cement composition of the present invention comprises a liquid component including ethyl methacrylate as the main component and a powder component including an ethyl methacrylate/methyl methacrylate copolymer as the main component.

### BACKGROUND OF THE INVENTION

1. Field of the Invention

The present invention relates to bone cements. More specifically, the present invention relates to bone cement compositions comprising a liquid component including ethyl methacrylate as the main component.

2. Description of the Prior Art

Bone cements are widely used to fix artificial joints. Bone cements are generally constituted by a liquid component including methyl methacrylate as the main component and a powder component including a homopolymer or a copolymer of methyl methacrylate. In the process of a surgical operation, the liquid component and the powder component are mixed, so that N,N-dimethyl-p-toluidine and benzoyl peroxide that are contained in the respective components are reacted and curing proceeds, which produces a cured cement in a short time and thus an artificial joint can be fixed (e.g., see Japanese Patent Publication (Tokko) No. 5-88147, Japanese Laid-Open Patent Publication (Tokuhyo) Nos. 62-500499, 3-502539, and (Tokkai) No. 4-189363). In order to make it easy to check the state of the cured cement in a human body, in the bone cement, in general, an X-ray contrast medium such as barium sulfate or zirconium oxide is added to the powder component.

Conventionally, the bone cement as described above has been used in human bodies for a long time, but there are the following problems. First, the exothermic reaction occurs rapidly when the liquid component and the powder component are mixed as the polymerization progresses. The cement temperature may reach near 100° C., and therefore it is pointed out that this high temperature injures tissues. Secondly, when the liquid component and the powder component are mixed, the mixture is changed rapidly from a muddy state to a dough state, and finally a solid state. The viscosity of the mixture affects the joint fixing operation, whether the viscosity is low or high. Therefore, when the mixture is applied to a human body at a wrong timing, satisfactory results cannot be obtained. In other words, it is necessary to perform a joint fixing operation within a time when the mixture has an appropriate viscosity. This time is referred to as “working time”. However, since the time during which the mixture can have an appropriate viscosity is short, it is necessary to perform bothering operations such as cooling the liquid component in order to prolong the working time. Thirdly, insertion of a bone cement causes a blood pressure drop temporarily, and in the worst case, the patient may die.

### SUMMARY OF THE INVENTION

It is an object of the present invention to provide a bone cement composition that has a low heat generation and a long working time, and is safer without a hypotensive action.

The inventors have found that the above-described problem can be solved by using a component different from conventionally used components as the main components of the liquid component and the powder component.

The present invention provides a bone cement composition comprising a liquid component including ethyl methacrylate as a main component and a powder component including an ethyl methacrylate/methyl methacrylate copolymer as a main component.

In a preferable embodiment, the copolymer comprises 20 to 80 wt % of ethyl methacrylate and 20 to 80 wt % of methyl methacrylate.

In a further preferable embodiment, the copolymer comprises 30 to 70 wt % of ethyl methacrylate and 30 to 70 wt % of methyl methacrylate.

In a still further preferable embodiment, the copolymer is a spherical powder having an average particle size of 20 to 70 μm, a weight-average molecular weight of the copolymer is 1×10⁵ to 5×10⁵, and the powder component further contains benzoyl peroxide in a ratio of 0.8 to 3 wt %.

In a further preferable embodiment, the copolymer is a spherical powder having an average particle size of 30 to 50 μm.

In a further preferable embodiment, the weight-average molecular weight of the copolymer is 1×10⁵ to 3×10⁵.

In a further preferable embodiment, the powder component further contains benzoyl peroxide in a ratio of 1 to 2.5 wt %.

In another preferable embodiment, the liquid component further contains a polymerization accelerator and/or a polymerization inhibitor.

In a further preferable embodiment, the powder component further contains an X-ray contrast medium.

According to the present invention, a bone cement composition having a lower heat generation than that of conventional products is provided. Therefore, tissue injury due to high temperature can be reduced. The bone cement composition of the present invention can be adjusted to have a long working time and an appropriate curing time by adjusting the copolymerization ratio of ethyl methacrylate/methyl methacrylate, the particle size and the weight-average molecular weight of the powder component. Therefore, the surgical operation using bone cements such as artificial joint operation can be performed safely and reliably. Furthermore, the bone cement composition of the present invention affects a human body less than the conventional bone cements in which the liquid component is based on methyl methacrylate, because the bone cement composition of the present invention is based on ethyl methacrylate whose toxicity in monomer is low. Moreover, the present invention is advantageous in that polymerization shrinkage occurs less in ethyl methacrylate, as generally known.

### DESCRIPTION OF THE PREFERRED EMBODIMENTS

The bone cement composition of the present invention is constituted by a liquid component including ethyl methacrylate as the main component and a powder component including an ethyl methacrylate/methyl methacrylate copolymer as the main component.

In the composition of the present invention, the liquid component includes ethyl methacrylate as the main component. Other than ethyl methacrylate, polymerization accelerators and polymerization inhibitors as described below, and minor components (e.g., pigment) can be contained.

In general, polymerization accelerators for accelerating degradation of benzoyl peroxide to produce radicals and polymerization inhibitors for ensuring the storage stability are added to the liquid component. As the polymerization accelerators, polymerization accelerators commonly used by those skilled in the art can be used. For example, N,N-dimethyl-p-toluidine, diethanol-p-toluidine, or other aromatic tertiary amines can be used, and added in a ratio of 0.5 to 3 percent by weight (hereinafter referred to as wt %). The larger this amount is, the shorter the working time and the curing time are, and the higher the maximum temperature during exothermic reaction is, so that 1 to 2 wt % is preferable. As the polymerization inhibitor, typically, 50 to 100 ppm of hydroquinone is used.

In the composition of the present invention, the powder component includes a copolymer of 20 to 80 wt % of ethyl methacrylate and 20 to 80 wt % of methyl methacrylate as the main component. Preferably, this copolymer is a spherical powder having an average particle size of 20 to 70 μm, and the weight-average molecular weight is 1×10⁵ to 5×10⁵. The powder component generally contains benzoyl peroxide. The characteristics of these powder components significantly affect the working time, the curing time and the exothermic reaction time of the cement mixture.

As described above, the copolymerization ratio of the ethyl methacrylate/methyl methacrylate copolymer is preferably 20/80 to 80/20 in weight ratio. The larger the ratio of ethyl methacrylate is, the shorter the working time and the curing time are, and the higher the maximum temperature during exothermic reaction is. Therefore, it is more preferable that the ratio is 30/70 to 70/30. The weight-average molecular weight of the copolymer is preferably 1×10⁵ to 5×10⁵. The higher the molecular weight is, the shorter the working time tends to be, the longer the curing time tends to be, and the lower the maximum temperature during exothermic reaction tends to be. Therefore, it is more preferable that the weight-average molecular weight is 1×10⁵ to 3×10⁵. The copolymer is preferably a spherical powder having an average particle size of 20 to 70 μm. The larger the particle size is, the longer the working time and the curing time tend to be, and the lower the exothermic reaction time tends to be. Therefore, a particle size of about 30 to 50 μm is more preferable. The powder shape is basically spherical, but non-spherical particles, cracked spherical particles, or crushed particles can be mixed.

The content ratio of benzoyl peroxide, which is a powder component, is preferably 0.8 to 3 wt %. The higher the content ratio is, the shorter the working time and the curing time tend to be, and the higher the exothermic reaction time tends to be, so that about 1 to 2.5 wt % is more preferable.

An X-ray contrast medium can be contained in the powder component for examination with X rays after insertion to a human body. As the X-ray contrast medium, known X-ray contrast media such as barium sulfate and zirconium oxide can be used, and generally contained in a ratio of about 10 wt %. The X-ray contrast medium can be contained up to about 30 wt % or up to about 50 wt %, if necessary.

The mixing ratio of the liquid component and the powder component is typically 20 g of the powder component with respect to 10 ml of the liquid component. It is preferable that the exothermic reaction time is as short as possible. A working time of 5 to 7 minutes and a curing time of 8 to 13 minutes are appropriate. If the working time is too short, it becomes difficult to insert the artificial joint to a human body safely and reliably. On the other hand, if the working time is too long, the curing time becomes inevitably long. If the curing time is too short, an artificial joint cannot be inserted reliably and on the other hand, too long a time is disadvantageous in that the time during which the surgeon holds and fixes the artificial joint becomes long.

In the present invention, the working time, the curing time and the exothermic reaction time are defined as follows.

Working time: The time during which the liquid component and the powder component are mixed in a predetermined ratio in a small glass container, and the mixture is fed to a 2 ml syringe and is ready to be pushed out easily from the syringe by hand is defined as the working time.

Curing time and maximum temperature during exothermic reaction: The same mixture as in the case of measuring the working time is prepared, and then this mixture is fed to a small polypropylene container at 37° C. A temperature sensor is inserted in its central portion and a temperature change is recorded. The time at which the temperature shows the peak after the start of mixing is defined as the curing time, and the temperature at that time is defined as the maximum temperature during exothermic reaction.

Next, the present invention will be described more specifically by way of examples.

### EXAMPLES

### Example 1

First, 1 ml of a liquid component including ethyl methacrylate containing 1 wt % of N,N-dimethyl-p-toluidine and a powder component including 1.8 g of a spherical powder of ethyl methacrylate/methyl methacrylate (30/70) copolymer having an average particle size of 30 μm and a weight-average molecular weight of 2.6×10⁵ containing 1.8% benzoyl peroxide and 0.2 g of barium sulfate were mixed sufficiently in a small glass container to prepare a mixture. Then, the working time, the curing time, and the maximum temperature during exothermic reaction for this mixture were measured in the following manner. For the working time, the mixture was fed to a 2 ml syringe and the time at which the mixture could be pushed out easily by hand was measured. For the curing time and the maximum temperature during exothermic reaction, the mixture was fed to a small polypropylene container, a temperature sensor was inserted in its central portion, and a temperature change was recorded at 37° C. The time at which the temperature showed the peak after the start of mixing (curing time) and the temperature at the peak (maximum temperature during exothermic reaction) were measured. The results are shown in Table 1.

### Examples 2 to 15

The working time, the curing time, and the maximum temperature during exothermic reaction were measured in the same manner as in Example 1 except that the concentration of N,N-dimethyl-p-toluidine in the liquid component, and the copolymerization ratio of ethyl methacrylate/methyl methacrylate, the particle size, the weight-average molecular weight and the content ratio of benzoyl peroxide in the powder component were changed to the values shown in Table 1. The results are shown in Table 1.

### Comparative Example 1

First, 1 ml of a liquid component including methyl methacrylate containing 1 wt % of N,N-dimethyl-p-toluidine and a powder component including 1.8 g of a spherical powder of polymethyl methacrylate having an average particle size of 45 μm and a weight-average molecular weight of 1.9×10⁵ containing 1.0% benzoyl peroxide and 0.2 g of barium sulfate were mixed sufficiently in a small glass container to prepare a mixture. Then, regarding this mixture, the working time, the curing time, and the maximum temperature during exothermic reaction were measured in the same manner as in Example 1. The results are shown in Table 1.

### Comparative Examples 2 to 4

The working time, the curing time, and the maximum temperature during exothermic reaction were measured in the same manner as in Example 1, except that methyl methacrylate containing 1 wt % of N,N-dimethyl-p-toluidine was used as the liquid component, and the copolymerization ratio of ethyl methacrylate/methyl methacrylate, the particle size, the weight-average molecular weight and the content ratio of benzoyl peroxide in the powder component were changed to the values shown in Table 1. The results are shown in Table 1.

### Comparative Example 5

The working time, the curing time, and the maximum temperature during exothermic reaction were measured in the same manner as in Example 1, using a commercially available bone cement (Osteobond manufactured by Zimmer Inc.; containing about 10 wt % of barium sulfate) which contains the liquid component and the powder component in the amounts shown in Table 1. The results are shown in Table 1.

### Comparative Example 6

The working time, the curing time, and the maximum temperature during exothermic reaction were measured in the same manner as in Example 1, using a commercially available bone cement (Surgical Simplex P manufactured by Howmedica Inc.; containing about 10 wt % of barium sulfate) which contains the liquid component and the powder component in the amounts shown in Table 1. The results are shown in Table 1.

TABLE 1

| | | | | | | | Measurement item | | |
|---|---|---|---|---|---|---|---|---|---|
| | Liquid component | | Powder component | | | | | | Maximum |
| | | N,N- | | | Weight- | | | | temperature |
| | | dimethyl- | | | average | Benzoyl | | | during |
| | | p-toluidine | EMA/MMA | Particle | molecular | peroxide | Working | Curing | exothermic |
| | Monomer | concentration | copolymerization | size | weight | content | time | time | reaction |
| | type | (%) | ratio | (μm) | (×10⁵) | (%) | (min) | (min) | (° C.) |
| Ex. 1 | EMA | 1 | 30/70 | 30 | 2.6 | 1.8 | 7.0 | 13.0 | 65 |
| Ex. 2 | EMA | 1 | 30/70 | 30 | 1.5 | 1.8 | 7.0 | 13.0 | 55 |
| Ex. 3 | EMA | 2 | 30/70 | 30 | 1.5 | 1.8 | 7.0 | 12.0 | 58 |
| Ex. 4 | EMA | 1 | 50/50 | 40 | 3.0 | 1.5 | 5.5 | 10.0 | 70 |
| Ex. 5 | EMA | 1 | 50/50 | 40 | 1.7 | 1.5 | 6.5 | 11.0 | 67 |
| Ex. 6 | EMA | 2 | 50/50 | 40 | 1.7 | 1.5 | 6.0 | 10.5 | 69 |
| Ex. 7 | EMA | 1 | 70/30 | 50 | 3.3 | 1.2 | 5.0 | 9.0 | 70 |
| Ex. 8 | EMA | 1 | 70/30 | 50 | 2.0 | 1.2 | 5.5 | 11.0 | 68 |
| Ex. 9 | EMA | 2 | 70/30 | 50 | 2.0 | 1.2 | 5.0 | 10.5 | 70 |
| Ex. 10 | EMA | 1 | 10/90 | 40 | 2.5 | 1.8 | 6.0 | 22.0 | 55 |
| Ex. 11 | EMA | 1 | 50/50 | 30 | 10.6 | 1.4 | 4.0 | 11.0 | 72 |
| Ex. 12 | EMA | 1 | 90/10 | 59 | 3.4 | 1.8 | 3.0 | 8.5 | 71 |
| Ex. 13 | EMA | 2 | 30/70 | 104 | 2.1 | 1.1 | 7.0 | 15.5 | 59 |
| Ex. 14 | EMA | 1 | 20/80 | 30 | 2.3 | 1.7 | 7.5 | 18.0 | 57 |
| Ex. 15 | EMA | 1 | 80/20 | 35 | 2.8 | 1.6 | 3.5 | 9.0 | 70 |
| Com. | MMA | 1 | 0/100 | 45 | 1.9 | 1.0 | 4.5 | 8.5 | 91 |
| Ex. 1 | | | | | | | | | |
| Com. | MMA | 1 | 30/70 | 30 | 2.6 | 1.8 | 4.0 | 8.0 | 90 |
| Ex. 2 | | | | | | | | | |
| Com. | MMA | 1 | 50/50 | 40 | 1.7 | 1.5 | 3.5 | 8.0 | 92 |
| Ex. 3 | | | | | | | | | |
| Com. | MMA | 1 | 70/30 | 50 | 2.0 | 1.2 | 2.5 | 8.0 | 92 |
| Ex. 4 | | | | | | | | | |
| Com. | MMA | 0.75 | 0/100 | 33 | 3.4 | 0.8 | 3.5 | 8.0 | 85 |
| Ex. 5* | | | | | | | | | |
| Com. | MMA | 2.6 | 0/100 | 41 | 2.2 | 1.2 | 3.0 | 8.0 | 91 |
| Ex. 6* | | | | | | | | | |

EMA: ethyl methacrylate; MMA: methyl methacrylate
*commercially available bone cement

In all of the Examples 1 to 15, the maximum temperatures during exothermic reaction are lower than that of the Comparative Examples 1 to 6. This shows that when the bone cement composition of the present invention is applied, the tissues around the applied site are injured less. The working time and the curing time can be longer than those of the conventional products by adjusting the copolymerization ratio of ethyl methacrylate/methyl methacrylate, the particle size and the weight-average molecular weight as appropriate in the present composition.

### Reference Example 1

Regarding ethyl methacrylate and methyl methacrylate, monomers thereof were injected into the femur of a rat in amounts of 0.01 ml, 0.02 ml, and 0.03 ml per kg of the weight of the rat, and the blood pressure drop effect was compared. For methyl methacrylate, the pressure was dropped more significantly in 0.02 ml injection, and the rat died from pressure drop in 0.03 ml injection. For ethyl methacrylate, the pressure drop was observed in 0.03 ml injection, but the rat was alive. Thus, it was confirmed that the pressure drop effect in ethyl methacrylate was far weaker than in methyl methacrylate. Therefore, it can be inferred that the pressure drop effect of the bone cement based on ethyl methacrylate as the liquid component is much weaker than that based on methyl methacrylate.

The invention may be embodied in other forms without departing from the spirit or essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

What is claimed is:

1. A bone cement composition comprising a liquid component including ethyl methacrylate as a main component and a powder component including an ethyl methacrylate/methyl methacrylate copolymer as a main component.

2. The bone cement composition of claim 1, wherein the copolymer comprises 20 to 80 wt % of ethyl methacrylate and 20 to 80 wt % of methyl methacrylate.

3. The bone cement composition of claim 2, wherein the copolymer comprises 30 to 70 wt % of ethyl methacrylate and 30 to 70 wt % of methyl methacrylate.

4. The bone cement composition of claim 1, wherein the copolymer is a spherical powder having an average particle size of 20 to 70 μm, a weight-average molecular weight of the copolymer is 1×10⁵ to 5×10⁵, and the powder component further contains benzoyl peroxide in a ratio of 0.8 to 3 wt %.

5. The bone cement composition of claim 4, wherein the copolymer is a spherical powder having an average particle size of 30 to 50 μm.

6. The bone cement composition of claim 4, wherein the weight-average molecular weight of the copolymer is 1×10⁵ to 3×10⁵.

7. The bone cement composition of claim 4, wherein the powder component further contains benzoyl peroxide in a ratio of 1 to 2.5 wt %.

8. The bone cement composition of claim 1, wherein the liquid component further contains a polymerization accelerator and/or a polymerization inhibitor.

9. The bone cement composition of claim 1, wherein the powder component further contains an X-ray contrast medium.

10. The bone cement composition of claim 2, wherein the copolymer is a spherical powder having an average particle size of 20 to 70 μm, a weight-average molecular weight of the copolymer is 1×10⁵ to 5×10⁵, and the powder component further contains benzoyl peroxide in a ratio of 0.8 to 3 wt %.

11. The bone cement composition of claim 2, wherein the liquid component further contains a polymerization accelerator and/or a polymerization inhibitor.

12. The bone cement composition of claim 2, wherein the powder component further contains an X-ray contrast medium.

13. The bone cement composition of claim 3, wherein the copolymer is a spherical powder having an average particle size of 20 to 70 μm, a weight-average molecular weight of the copolymer is 1×10⁵ to 5×10⁵, and the powder component further contains benzoyl peroxide in a ratio of 0.8 to 3 wt %.

14. The bone cement composition of claim 3, wherein the liquid component further contains a polymerization accelerator and/or a polymerization inhibitor.

15. The bone cement composition of claim 3, wherein the powder component further contains an X-ray contrast medium.

16. The bone cement composition of claim 4, wherein the liquid component further contains a polymerization accelerator and/or a polymerization inhibitor.

17. The bone cement composition of claim 4, wherein the powder component further contains an X-ray contrast medium.

18. The bone cement composition of claim 8, wherein the powder component further contains an X-ray contrast medium.
